SEQUENCE LISTING (4 sequences)

SEQ ID NO: 1 - DNA, 1842 bp, Penicillium russellii
atgcgctatactcttttaacgagtattgccagcgtccttagcgtggggccgctggcatctgcgagccctacatcgaaggatggaaatttggcatcctatatagcgaaagagggacaacgatctattgtggggatcactgaaaacctcggtggtaaaggcagcaagacaccgggcacggccgctggcttattcattgctagtcctaatatggcgaaccccaattactattatacgtggactcgtgattcggcccttacattcaaatgcttgatcgacctgtttgaaacttctgatcaggactatatcagtcgtaaagagttggaaaccgacattcggaattatgtctcatcacaagcagtgctgcagaatgtatccaatccatctgggactctgaaagatggctctggactcggtgagcccaagtttgagatcgacctgaacccattcagtggttcctggggccggcctcagcgtgacggtcccgccctgcgagcgaccgccatgatcacttacgcagactggttggtttcccacggtcagaaatccgaagccaccaacatcatgtggcccatcattgcaaatgacttggcatatgtaggccagtactggaacaaaactggattcgacctctgggaagaggtagatggatcttccttttacaccttggctgtccaacaccgagcactggtccagggagcaagccttgcgaagaagcttggcaagtcgtgcactgcttgtgtatcccaagcacctcagattctttgctttctccagagcttctggaacggaaattacatcaccgccaacatcaatcttgacacaagtcgctccggtatcgatttgaactcgatcttgggaagcatccacacctttgatcccgaagcttcatgtgatgattcgaccttccaaccttgctcagccagagcgcttgcaaaccataaggtctatgttgacgcgttccgttcaatctatggtgtcaatgctggcctttcaaacgggacagccgccaatgtcggtcggtacccagaagatgtttaccaaggaggtaacccatggtatctcgccactctagcagcagctgaactgctgtacgatgctctataccaatggaaccaaattggcaagctcgatgtcaccaagacctcgctagcgttcttcaaagattttgacgcggccgtcaaaacaggcacatactcagcgcatagctcagcctacaggactctcacctcagccatccgaacttatgcagatgatttcatcagtctcgtccaacactatactccttctaatgggtccttggccgagcaatatgatcgggatactggtatcccactgtcagccaatgatctaacttggtcttacgcttccttcatcacagctatcgagcgtcgtgcttccgtcgtgcccgcctcatggggcgaaaaatctgcaaatgtggttcccactacctgctcagcctctcctgtcactggaacttacgtagctgctacttcagtattcccgacaaccactggatgtgttccggcgacaagcatcgttccgatcacattctacttgactgagagcacattctatggagaaaatgtctacatgactggcaacattagtgcacttggcaactgggacacgagcagtggtttccccctcacagcaaacttatacactgattcagaccatttgtggtttgccagtgttgagcttgttccggcggggacaccgtttgaatacaaatactacaaggtagagccgaatggtactgtcatttgggagaacggtgagaacagggtatacgttgctcctactgggtgtccgatccagcctagccaaaccgatatttggcgt

SEQ ID NO: 2 - protein, 614 aa, Penicillium russellii
MRYTLLTSIASVLSVGPLASASPTSKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 3 - protein, 590 aa, Penicillium russellii
SKDGNLASYIAKEGQRSIVGITENLGGKGSKTPGTAAGLFIASPNMANPNYYYTWTRDSALTFKCLIDLFETSDQDYISRKELETDIRNYVSSQAVLQNVSNPSGTLKDGSGLGEPKFEIDLNPFSGSWGRPQRDGPALRATAMITYADWLVSHGQKSEATNIMWPIIANDLAYVGQYWNKTGFDLWEEVDGSSFYTLAVQHRALVQGASLAKKLGKSCTACVSQAPQILCFLQSFWNGNYITANINLDTSRSGIDLNSILGSIHTFDPEASCDDSTFQPCSARALANHKVYVDAFRSIYGVNAGLSNGTAANVGRYPEDVYQGGNPWYLATLAAAELLYDALYQWNQIGKLDVTKTSLAFFKDFDAAVKTGTYSAHSSAYRTLTSAIRTYADDFISLVQHYTPSNGSLAEQYDRDTGIPLSANDLTWSYASFITAIERRASVVPASWGEKSANVVPTTCSASPVTGTYVAATSVFPTTTGCVPATSIVPITFYLTESTFYGENVYMTGNISALGNWDTSSGFPLTANLYTDSDHLWFASVELVPAGTPFEYKYYKVEPNGTVIWENGENRVYVAPTGCPIQPSQTDIWR

SEQ ID NO: 4 - DNA, 1845 bp, Penicillium russellii
atgcgctatactcttttaacgagtattgccagcgtccttagcgtggggccgctggcatctgcgagccctacatcgaaggatggaaatttggcatcctatatagcgaaagagggacaacgatctattgtggggatcactgaaaacctcggtggtaaaggcagcaagacaccgggcacggccgctggcttattcattgctagtcctaatatggcgaaccccaattactattatacgtggactcgtgattcggcccttacattcaaatgcttgatcgacctgtttgaaacttctgatcaggactatatcagtcgtaaagagttggaaaccgacattcggaattatgtctcatcacaagcagtgctgcagaatgtatccaatccatctgggactctgaaagatggctctggactcggtgagcccaagtttgagatcgacctgaacccattcagtggttcctggggccggcctcagcgtgacggtcccgccctgcgagcgaccgccatgatcacttacgcagactggttggtttcccacggtcagaaatccgaagccaccaacatcatgtggcccatcattgcaaatgacttggcatatgtaggccagtactggaacaaaactggattcgacctctgggaagaggtagatggatcttccttttacaccttggctgtccaacaccgagcactggtccagggagcaagccttgcgaagaagcttggcaagtcgtgcactgcttgtgtatcccaagcacctcagattctttgctttctccagagcttctggaacggaaattacatcaccgccaacatcaatcttgacacaagtcgctccggtatcgatttgaactcgatcttgggaagcatccacacctttgatcccgaagcttcatgtgatgattcgaccttccaaccttgctcagccagagcgcttgcaaaccataaggtctatgttgacgcgttccgttcaatctatggtgtcaatgctggcctttcaaacgggacagccgccaatgtcggtcggtacccagaagatgtttaccaaggaggtaacccatggtatctcgccactctagcagcagctgaactgctgtacgatgctctataccaatggaaccaaattggcaagctcgatgtcaccaagacctcgctagcgttcttcaaagattttgacgcggccgtcaaaacaggcacatactcagcgcatagctcagcctacaggactctcacctcagccatccgaacttatgcagatgatttcatcagtctcgtccaacactatactccttctaatgggtccttggccgagcaatatgatcgggatactggtatcccactgtcagccaatgatctaacttggtcttacgcttccttcatcacagctatcgagcgtcgtgcttccgtcgtgcccgcctcatggggcgaaaaatctgcaaatgtggttcccactacctgctcagcctctcctgtcactggaacttacgtagctgctacttcagtattcccgacaaccactggatgtgttccggcgacaagcatcgttccgatcacattctacttgactgagagcacattctatggagaaaatgtctacatgactggcaacattagtgcacttggcaactgggacacgagcagtggtttccccctcacagcaaacttatacactgattcagaccatttgtggtttgccagtgttgagcttgttccggcggggacaccgtttgaatacaaatactacaaggtagagccgaatggtactgtcatttgggagaacggtgagaacagggtatacgttgctcctactgggtgtccgatccagcctagccaaaccgatatttggcgttaa

(19) United States
(12) Patent Application Publication
(10) Pub. No.: US 2023/0002749 A1
(43) Pub. Date: Jan. 5, 2023

(54) THERMOSTABLE GLUCOAMYLASE AND METHODS OF USE, THEREOF

(71) Applicant: Danisco US Inc., Palo Alto, CA (US)

(72) Inventors: Jacob Flyvholm Cramer, Palo Alto, CA (US); Jing Ge, Palo Alto, CA (US); Marc Anton Bernhard Kolkman, Palo Alto, CA (US); Igor Nikolaev, Palo Alto, CA (US); Jayarama K. Shetty, Palo Alto, CA (US); Zhongmei Tang, Palo Alto, CA (US); Wilhelmus Antonius Van Der Kleij, Palo Alto, CA (US); Zhiyong Xie, Palo Alto, CA (US); Bo Zhang, Palo Alto, CA (US); Zhengzheng Zou, Palo Alto, CA (US)

(21) Appl. No.: 16/491,078

(22) PCT Filed: Nov. 9, 2017
(86) PCT No.: PCT/US17/60744

§ 371 Date: Sep. 4, 2019

(30) Foreign Application Priority Data
Mar. 7, 2017 (CN) PCT/CN2017/075872

(51) Int. Cl. C12N 9/34; C12C 5/00

(52) U.S. Cl. CPC C12N 9/2428; C12C 5/004; C12Y 302/01003

(57) ABSTRACT

Described are polypeptides having glucoamylase activity, compositions comprising such polypeptides, and methods of using such polypeptides and compositions.

### CROSS REFERENCE TO RELATED APPLICATION

This application claims the benefit of International Patent Application No. PCT/CN2013/076419, filed 7 Mar. 2017, which is herein incorporated by reference in its entirety.

### FIELD OF THE INVENTION

The present disclosure relates to polypeptides having glucoamylase activity and compositions comprising such polypeptides. The present disclosure further relates to polynucleotides encoding such polypeptides, vectors and host cells comprising genes encoding such polypeptides, which may also enable the production of such polypeptides. The disclosure also relates to methods of saccharifying starch-containing materials using or applying the polypeptides or compositions, as well as the saccharides thus produced by the method. Moreover, the disclosure relates to methods of producing fermentation products as well as the fermentation products produced by the method thereof.

### BACKGROUND

Glucoamylase (1,4-alpha-D-glucan glucohydrolase, EC 3.2.1.3) is an enzyme, which catalyzes the release of D-glucose from the non-reducing ends of starch or related oligo- and poly-saccharide molecules. Glucoamylases are produced by several filamentous fungi and yeast.

The major application of glucoamylase is the saccharification of partially processed starch/dextrin to glucose, which is an essential substrate for numerous fermentation processes. The glucose may then be converted directly or indirectly into a fermentation product using a fermenting organism. Examples of commercial fermentation products include alcohols (e.g., ethanol, methanol, butanol, 1,3-propanediol); organic acids (e.g., citric acid, acetic acid, itaconic acid, lactic acid, gluconic acid, gluconate, lactic acid, succinic acid, 2,5-diketo-D-gluconic acid); ketones (e.g., acetone); amino acids (e.g., glutamic acid); gases (e.g., H₂ and CO₂), and more complex compounds, including, for example, antibiotics (e.g., penicillin and tetracycline); enzymes; vitamins (e.g., riboflavin, B₁₂, beta-carotene); hormones, and other compounds which are difficult to produce synthetically. Fermentation processes are also commonly used in the consumable alcohol (e.g., beer and wine), dairy (e.g., in the production of yogurt and cheese), leather, beverage and tobacco industries.

The end product may also be syrup. For instance, the end product may be glucose, but may also be converted, e.g., by glucose isomerase to fructose or a mixture composed almost equally of glucose and fructose. This mixture, or a mixture further enriched with fructose, is the most commonly used high fructose corn syrup (HFCS) commercialized throughout the world.

Glucoamylase for commercial purposes has traditionally been produced employing filamentous fungi, although a diverse group of microorganisms is reported to produce glucoamylase, since they secrete large quantities of the enzyme extracellularly. However, the commercially used fungal glucoamylases have certain limitations such as moderate thermostability, acidic pH requirement, and slow catalytic activity that increase the process cost. Therefore, there is a need to search for new glucoamylases to improve temperature optima leading to amelioration in catalytic efficiency to shorten the saccharification time or get higher yield of end products.

It is an object of the present disclosure to provide certain polypeptides having glucoamylase activity, polynucleotides encoding the polypeptides, nucleic acid constructs that can be used to produce such polypeptides, compositions comprising thereof, as well as methods of applying such polypeptides to different industial applications.

### SUMMARY

The present polypeptides, compositions and methods of saccharifying starch-containing materials using or applying the polypeptides or compositions. Aspects and embodiments of the polypeptides, compositions and methods are described in the following, independently-numbered paragraphs.
1. In one aspect, a polypeptide having glucoamylase activity, selected from the group consisting of:
   (a) a polypeptide comprising an amino acid sequence having at least 95%, such as even at least 96%, 97%, 98%, 99% or 100% identity to the polypeptide of SEQ ID NO: 3;
   (b) a polypeptide encoded by a polynucleotide that hybridizes under at least low stringency conditions, more preferably at least medium stringency conditions, even more preferably at least medium-high stringency conditions, most preferably at least high stringency, and even most preferably at least very high stringency conditions with
      (i) the mature polypeptide coding sequence of SEQ ID NO: 1,
      (ii) the genomic DNA sequence comprising the mature polypeptide coding sequence of SEQ ID NO: 1, or
      (iii) a full-length complementary strand of (i) or (ii);
   (c) a polypeptide encoded by a polynucleotide comprising a nucleotide sequence having preferably at least 75%, more preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, 97%, 98%, 99% or 100% identity to the mature polypeptide coding sequence of SEQ ID NO: 1;
   (d) a variant comprising a substitution, deletion, and/or insertion of one or more (e.g., several) amino acids of the mature polypeptide of SEQ ID NO: 2; and
   (e) a fragment of a polypeptide of (a), (b), (c) or (d) that has glucoamylase activity.
2. In another aspect, a polynucleotide comprising a nucleotide sequence that encodes the polypeptide of paragraph 1.
3. In some embodiments of the polynucleotide of paragraph 2 operably linked to one or more control sequences that control the production of the polypeptide in an expression host.
4. In another aspect, a recombinant host cell comprising the polynucleotide of paragraph 2.
5. In some embodiments of the host cell of paragraph 4, which is an ethanologenic microorganism.
6. In some embodiments of the host cell of paragraph 4 or 5, which further expresses and secretes one or more additional enzymes selected from the group comprising protease, hemicellulase, cellulase, peroxidase, lipolytic enzyme, metallolipolytic enzyme, xylanase, lipase, phospholipase, esterase, perhydrolase, cutinase, pectinase, pectate lyase, mannanase, keratinase, reductase, oxidase, phenoloxidase, lipoxygenase, ligninase, alpha-amylase, pullulanase, phytase, tannase, pentosanase, malanase, beta-glucanase, arabinosidase, hyaluronidase, chondroitinase, laccase, transferrase, or a combination thereof.
7. In another aspect, a method for saccharifying a starch-containing material comprising the steps of: i) contacting the starch-containing material with an alpha-amylase; and ii) contacting the starch-containing material with a glucoamylase at a temperature of at least 70° C.; wherein the method produces at least 70% free glucose from the starch-containing material (substrate).
8. In some embodiments of the method of paragraph 7, wherein the step (ii) is carried out at a temperature of at least 75° C., preferably at least 80° C. for between 12 and 96 hours, preferably 18 to 60 hours.
9. In some embodiments of the method of paragraph Tor 8, wherein the glucoamylase maintains at least 70%, at least 80%, at least 85%, at least 90%, at least 95%, at least 100% of relative activity at a temperature of at least 70° C., and/or at a pH between 2.0 and 7.0, preferably between pH 4.0 and pH 6.0, more preferably between pH 4.5 and pH 5.5.
10. In some embodiments of the method of any of paragraphs 7-9, wherein the method includes sequentially or simultaneously performing step (i) and step (ii).
11. In some embodiments of the method of any of paragraphs 7-10, wherein the method further comprises a pre-saccharification before saccharification step ii).
12. In some embodiments of the method of any of paragraphs 7-11, wherein the glucoamylase is the polypeptide of claim 1.
13. In some embodiments of the method of any of paragraphs 7-12, wherein the step (i) is carried out at or below the gelatinization temperature of the starch-containing material.
14. In some embodiments of the method of any of paragraphs 7-13, wherein an additional debranching enzyme is absent during step (i) and/or step (ii).
15. In some embodiments of the method of paragraph 14, wherein the debranching enzyme is pullulanase.
16. In another aspect, a saccharide produced by method of any of paragraphs 7-15.
17. In another aspect, a method for producing fermentation products from the saccharide of paragraph 16, wherein the saccharide is fermented by a fermenting organism.
18. In some embodiments of the method of paragraph 17, wherein the fermentation process is performed sequentially or simultaneously with the step (ii).
19. In some embodiments of the method of paragraph 17 or 18, wherein the fermentation product comprises ethanol.
20. In some embodiments of the method of paragraph 17 or 18, wherein the fermentation product comprises a non-ethanol metabolite.
21. In some embodiments of the method of paragraph 20, wherein the metabolite is citric acid, lactic acid, succinic acid, monosodium glutamate, gluconic acid, sodium gluconate, calcium gluconate, potassium gluconate, an organic acid, glucono delta-lactone, sodium erythorbate, omega 3 fatty acid, butanol, iso-butanol, an amino acid, lysine, tyrosine, threonine, glycine, itaconic acid, 1,3-propanediol, vitamins, enzymes, hormones, isoprene or other biochemicals or biomaterials.
22. In another aspect, a method of applying the polypeptide of paragraph 1 in brewing.

### BRIEF DESCRIPTION OF THE DRAWINGS

FIG. 1 is plasmid map of pJG580.

FIG. 2 is production profile of PruGA1 over a 95-hour fermentation.

FIG. 3 is DP1 production comparison of PruGA1-0.3×, PruGA1-1×, AfuGA-1×, and AnGA-1× at 60, 65, and 70° C., after 72-h incubation.

FIG. 4 is comparison of activities towards raw starch of PruGA1 with TrGA at pH 3.5.

FIG. 5 is comparison of activities towards raw starch of PruGA1 with TrGA at pH 4.5.

### DETAILED DESCRIPTION

The present disclosure relates to polypeptides having glucoamylase activity and compositions comprising such polypeptides. The present disclosure further relates to polynucleotides encoding such polypeptides, vectors and host cells comprising genes encoding such polypeptides, which may also enable the production of such polypeptides. The disclosure also relates to methods of saccharifying starch-containing materials using or applying the polypeptides or compositions, as well as the saccharides thus produced by the method. Moreover, the disclosure relates to methods of producing fermentation products as well as the fermentation products produced by the method thereof.

Prior to describing the compositions and methods in detail, the following terms and abbreviations are defined.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

### Definition

The term “glucoamylase (1,4-alpha-D-glucan glucohydrolase, EC 3.2.1.3) activity” is defined herein as an enzyme activity, which catalyzes the release of D-glucose from the non-reducing ends of starch or related oligo- and poly-saccharide molecules.

The polypeptides of the present invention have at least 70%, more preferably at least 80%, even more preferably at least 90%, most preferably at least 95%, and even most preferably at least 100% of the glucoamylase activity of the mature polypeptide of SEQ ID NO: 2.

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “mature polypeptide” is defined herein as a polypeptide in its final form following translation and any post-translational modifications, such as N-terminal processing, C-terminal truncation, glycosylation, phosphorylation, etc. In one aspect, the mature polypeptide is amino acids 22 to 614 of SEQ ID NO: 2 based on the SignalP (Nielsen et al., 1997, Protein Engineering 10: 1-6) program that predicts amino acids 1 to 21 of SEQ ID NO: 2 are a signal peptide.

The term “nucleic acid” encompasses DNA, RNA, heteroduplexes, and synthetic molecules capable of encoding a polypeptide. Nucleic acids may be single stranded or double stranded, and may be chemically modified. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant nucleotide sequence.

The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

The term “hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ.

A “synthetic” molecule is produced by in vitro chemical or enzymatic synthesis rather than by an organism.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

The term “expression” refers to the process by which a polypeptide is produced based on a nucleic acid sequence. The process includes both transcription and translation.

The term “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refer to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

“Percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:

Gap opening penalty: 10.0

Gap extension penalty: 0.05

Protein weight matrix: BLOSUM series

DNA weight matrix: IUB

Delay divergent sequences %: 40

Gap separation distance: 8

DNA transitions weight: 0.50

List hydrophilic residues: GPSNDQEKR

Use negative matrix: OFF

Toggle Residue specific penalties: ON

Toggle hydrophilic penalties: ON

Toggle end gap separation penalty OFF.

The term “homologous sequence” is defined herein as a predicted protein having an E value (or expectancy score) of less than 0.001 in a tfasty search (Pearson, W. R., 1999, in Bioinformatics Methods and Protocols, S. Misener and S. A. Krawetz, ed., pp. 185-219) with the Penicillium russellii glucoamylase of SEQ ID NO: 3.

The term “polypeptide fragment” is defined herein as a polypeptide having one or more (e.g., several) amino acids deleted from the amino and/or carboxyl terminus of the polypeptide of SEQ ID NO: 3; or a homologous sequence thereof; wherein the fragment has glucoamylase activity.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

The terms “thermostable” and “thermostability,” with reference to an enzyme, refer to the ability of the enzyme to retain activity after exposure to an elevated temperature. The thermostability of an enzyme, such as an amylase enzyme, is measured by its half-life (t_{1/2}) given in minutes, hours, or days, during which half the enzyme activity is lost under defined conditions. The half-life may be calculated by measuring residual alpha-amylase activity for example following exposure to (i.e., challenge by) an elevated temperature.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “pre-saccharification” is defined herein as a process prior to the complete saccharification or simultaneous saccharification and fermentation (SSF). Pre-saccharification is carried out typically at a temperature between 30-65. deg. C, about 60. deg. C, for 40-90 minutes.

The phrase “simultaneous saccharification and fermentation (SSF)” refers to a process in the production of biochemicals in which a microbial organism, such as an ethanologenic microorganism, and at least one enzyme, such as an amylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

A “slurry” is an aqueous mixture containing insoluble starch granules in water.

The term “total sugar content” refers to the total soluble sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

“Dry solid content” refers to the percentage of dry solids both dissolved and dispersed as a percentage by weight with respect to the water in which the dry solids are dispersed and/or dissolved. The initial dry solid content of starch is the weight of granular starch corrected for moisture content over the weight of granular starch plus weight of water. Subsequent dry solid content can be determined from the initial content adjusted for any water added or lost and for chemical gain. Subsequent dissolved dry solid content can be measured from refractive index as indicated below 8.

The term “high DS” refers to aqueous starch slurry with a dry solid content greater than 38% (wt/wt).

“Dry substance starch” refers to the dry starch content of a substrate, such as a starch slurry, and can be determined by subtracting from the mass of the subtrate any contribution of non-starch components such as protein, fiber, and water. For example, if a granular starch slurry has a water content of 20% (wt/wt)., and a protein content of 1% (wt/wt), then 100 kg of granular starch has a dry starch content of 79 kg. Dry substance starch can be used in determining how many units of enzymes to use.

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4+(>DP3) denotes polymers with a degree of polymerization of greater than 3.

The term “contacting” refers to the placing of referenced components (including but not limited to enzymes, substrates, and fermenting organisms) in sufficiently close proximity to affect an expect result, such as the enzyme acting on the substrate or the fermenting organism fermenting a substrate. Those skilled in the art will recognize that mixing solutions can bring about “contacting.” An “ethanologenic microorganism” refers to a microorganism with the ability to convert a sugar or other carbohydrates to ethanol.

The term “biochemicals” refers to a metabolite of a microorganism, such as citric acid, lactic acid, succinic acid, monosodium glutamate, gluconic acid, sodium gluconate, calcium gluconate, potassium gluconate, glucono delta-lactone, sodium erythorbate, omega 3 fatty acid, butanol, iso-butanol, an amino acid, lysine, itaconic acid, other organic acids, 1,3-propanediol, vitamins, or isoprene or other biomaterial.

The term “pullulanase” also called debranching enzyme (E.C. 3.2.1.41, pullulan 6-glucanohydrolase), is capable of hydrolyzing alpha 1-6 glucosidic linkages in an amylopectin molecule.

The term “about” refers to ±15% to the referenced value.

The term “comprising” and its cognates are used in their inclusive sense; that is, equivalent to the term “including” and its corresponding cognates.

EC enzyme commission

CAZy carbohydrate active enzyme

w/v weight/volume

w/w weight/weight

v/v volume/volume

wt % weight percent

° C. degrees Centigrade

g or gm gram

μm microgram

mg milligram

kg kilogram

μL and μl microliter

mL and ml milliliter

mm millimeter

μm micrometer

mol mole

mmol millimole

M molar

mM millimolar

μM micromolar

nm nanometer

U unit

ppm parts per million

hr and h hour

EtOH ethanol

ds dry solid

Polypeptides Having glucoamylase Activity

In a first aspect, the present invention relates to polypeptides comprising an amino acid sequence having preferably at least 90%, more preferably at least 92%, even more preferably at least 93%, most preferably at least 94%, and even most preferably at least 95%, such as even at least 96%, 97%, 98%, 99% or 100% identity to the polypeptide of SEQ ID NO: 3, which have glucoamylase activity.

In some embodiments, the polypeptides of the present invention are the homologous polypeptides comprising amino acid sequences differ by ten amino acids, preferably by nine amino acids, preferably by eight amino acids, preferably by seven amino acids, preferably by six amino acids, preferably by five amino acids, more preferably by four amino acids, even more preferably by three amino acids, most preferably by two amino acids, and even most preferably by one amino acid from the polypeptide of SEQ ID NO: 3.

In some embodiments, the polypeptides of the present invention are the variants of polypeptide of SEQ ID NO: 3, or a fragment thereof having glucoamylase activity.

In some embodiments, the polypeptides of the present invention are thermostable and retain glucoamylase activity at increased temperature. The polypeptides of the present invention have shown thermostability at pH values ranging from about 2.5 to about 8.0 (e.g., about 3.0 to about 7.5, about 3.0 to about 7.0, about 3.0 to about 6.5, etc). For example, at pH of about 3.0 to about 7.0 (e.g., about 3.5 to about 6.5, etc), the polypeptides of the present invention retain most of glucoamylase activity for an extended period of time at high temperature (e.g., at least 50° C., at least 55° C., at least 60° C., at least 65° C., at least 70° C., at least 75° C., at least 80° C., at least 85° C., at least 90° C. or a higher temperature), for example, for at least 1 hour, at least 2 hours, at least 3 hours, at least 5 hours, or even longer. For example, the polypeptides of the present invention retain at least about 35% (e.g., at least about 35%, at least about 40%, at least about 45%, at least about 50%, at least about 55%, at least about 60%, at least about 65%, at least about 70% or a higher percentage) of glucoamylase activity when incubated for at least about 1 hours, 3 hours, 5 hours, or longer at increased temperature at a pH of from about 3.5 to about 6.5.

In some embodiments, the polypeptides of the present invention have maximum activity at a pH of about 5, have over 90% of maximum activity at a pH of about 3.5 to a pH of about 6.0, and have over 70% of maximum activity at a pH of about 2.8 to a pH of about 7.0, measured at a temperature of 50° C., as determined by the assays described, herein. Exemplary pH ranges for use of the enzyme are pH 2.5-7.0, 3.0-7.0, 3.5-7.0, 2.5-6.0, 3.0-6.0 and 3.5-6.0.

In some embodiments, the polypeptides of the present invention have maximum activity at a temperature of about 75° C., have over 70% of maximum activity at a temperature of about 63° C. to a temperature of about 79° C., measured at a pH of 5.0, as determined by the assays described, herein. Exemplary temperature ranges for use of the enzyme are 50-82° C., 50-80° C., 55-82° C., 55-80° C. and 60-80° C. In a second aspect, the present invention relates to polypeptides having glucoamylase activity that are encoded by polynucleotides that hybridize under preferably very low stringency conditions, more preferably low stringency conditions, more preferably medium stringency conditions, more preferably medium-high stringency conditions, even more preferably high stringency conditions, and most preferably very high stringency conditions with (i) the mature polypeptide coding sequence of SEQ ID NO: 1, (ii) the genomic DNA sequence comprising the mature polypeptide coding sequence of SEQ ID NO: 1, or (iii) a full-length complementary strand of (i) or (ii) (J. Sambrook, E. F. Fritsch, and T. Maniatis, 1989, Molecular Cloning, A Laboratory Manual, 2nd edition, Cold Spring Harbor, New York).

The nucleotide sequence of SEQ ID NO: 1; or a fragment thereof may be used to design nucleic acid probes to identify and clone DNA encoding polypeptides having glucoamylase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes that are preferably at least 600 nucleotides, more preferably at least 800 nucleotides, even more preferably at least 1000 nucleotides, even more preferably at least 1500 nucleotides, or most preferably at least 1800 nucleotides in length. Both DNA and RNA probes can be used. The probes are typically labeled for detecting the corresponding gene (for example, with ³²P, ³H, ³⁵S, biotin, or avidin). Such probes are also encompassed by the present invention.

A genomic DNA or cDNA library prepared from such other strains may, therefore, be screened for DNA that hybridizes with the probes described above and encodes a polypeptide having glucoamylase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1, or a subsequence thereof, the carrier material is preferably used in a Southern blot.

In a third aspect, the present invention relates to polypeptides having glucoamylase activity encoded by polynucleotides comprising nucleotide sequences having a degree of sequence identity to the mature polypeptide coding sequence of SEQ ID NO: 1 of preferably at least 60%, more preferably at least 63%, more preferably at least 65%, more preferably at least 68%, more preferably at least 70%, more preferably at least 72%, more preferably at least 75%, at least 77%, more preferably at least 79%, more preferably at least 81%, more preferably at least 83%, more preferably at least 85%, more preferably at least 90%, more preferably at least 92%, even more preferably at least 93%, most preferably at least 94%, and even most preferably at least 95%, such as even at least 96%, 97%, 98%, 99% or 100% identity, which encode a polypeptide having glucoamylase activity.

In a fourth aspect, the present glucoamylases comprise conservative substitution of one or several amino acid residues relative to the amino acid sequence of SEQ ID NO: 3. Exemplary conservative amino acid substitutions are listed in the Table 1. Some conservative mutations can be produced by genetic manipulation, while others are produced by introducing synthetic amino acids into a polypeptide by other means.

TABLE 1
Conservative amino acid substitutions

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S—Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S—Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some embodiments, the present glucoamylase comprises a deletion, substitution, insertion, or addition of one or a few amino acid residues relative to the amino acid sequence of SEQ ID NO: 3 or a homologous sequence thereof. In some embodiments, the present glucoamylases are derived from the amino acid sequence of SEQ ID NO: 3 by conservative substitution of one or several amino acid residues. In some embodiments, the present glucoamylases are derived from the amino acid sequence of SEQ ID NO: 3 by deletion, substitution, insertion, or addition of one or a few amino acid residues relative to the amino acid sequence of SEQ ID NO: 3. In all cases, the expression “one or a few amino acid residues” refers to 10 or less, i.e., 1, 2, 3, 4, 5, 6, 7, 8, 9, or 10, amino acid residues.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U. S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The amino acid substitutions, deletions and/or insertions of the mature polypeptide of SEQ ID NO: 2 can be at most 10, preferably at most 9, more preferably at most 8, more preferably at most 7, more preferably at most 6, more preferably at most 5, more preferably at most 4, even more preferably at most 3, most preferably at most 2, and even most preferably at most 1.

The glucoamylase may be a “chimeric” or “hybrid” polypeptide, in that it includes at least a portion from a first glucoamylase, and at least a portion from a second amylase, glucoamylase, beta-amylase, alpha-glucosidase or other starch degrading enzymes, or even other glycosyl hydrolases, such as, without limitation, cellulases, hemicellulases, etc. (including such chimeric amylases that have recently been “rediscovered” as domain-swap amylases). The present glucoamylases may further include heterologous signal sequence, an epitope to allow tracking or purification, or the like.

Production of glucoamylase

The present glucoamylases can be produced in host cells, for example, by secretion or intracellular expression. A cultured cell material (e.g., a whole-cell broth) comprising a glucoamylase can be obtained following secretion of the glucoamylase into the cell medium. Optionally, the glucoamylase can be isolated from the host cells, or even isolated from the cell broth, depending on the desired purity of the final glucoamylase. A gene encoding a glucoamylase can be cloned and expressed according to methods well known in the art. Suitable host cells include bacterial, fungal (including yeast and filamentous fungi), and plant cells (including algae). Particularly useful host cells include Aspergillus niger, Aspergillus oryzae, Trichoderma reesi or Myceliopthora Thermophila. Other host cells include bacterial cells, e.g., Bacillus subtilis or B. licheniformis, as well as Streptomyces.

Additionally, the host may express one or more accessory enzymes, proteins, peptides. These may benefit liquefaction, saccharification, fermentation, SSF, and downstream processes. Furthermore, the host cell may produce ethanol and other biochemicals or biomaterials in addition to enzymes used to digest the various feedstock(s). Such host cells may be useful for fermentation or simultaneous saccharification and fermentation processes to reduce or eliminate the need to add enzymes.

### Compositions

The present invention also relates to compositions comprising a polypeptide of the present invention. In some embodiments, a polypeptide comprising an amino acid sequence that is at least about 70%, at least about 75%, at least about 80%, at least about 85%, at least about 90%, at least about 95%, identical to that of SEQ ID NO: 1 can also be used in the enzyme composition. Preferably, the compositions are formulated to provide desirable characteristics such as low color, low odor and acceptable storage stability.

The composition may comprise a polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, alpha-glucosidase, beta-glucosidase, beta-amylase, isoamylase, haloperoxidase, invertase, laccase, lipase, mannosidase, oxidase, pectinolytic enzyme, peptidoglutaminase, peroxidase, phytase, polyphenoloxidase, proteolytic enzyme, pullulanase, ribonuclease, transglutaminase, xylanase or a combination thereof, which may be added in effective amounts well known to the person skilled in the art.

The polypeptide compositions may be prepared in accordance with methods known in the art and may be in the form of a liquid or a dry composition. For instance, the compositions comprising the present glucoamylases may be aqueous or non-aqueous formulations, granules, powders, gels, slurries, pastes, etc., which may further comprise any one or more of the additional enzymes listed, herein, along with buffers, salts, preservatives, water, co-solvents, surfactants, and the like. Such compositions may work in combination with endogenous enzymes or other ingredients already present in a slurry, water bath, washing machine, food or drink product, etc, for example, endogenous plant (including algal) enzymes, residual enzymes from a prior processing step, and the like. The polypeptide to be included in the composition may be stabilized in accordance with methods known in the art.

The composition may be cells expressing the polypeptide, including cells capable of producing a product from fermentation. Such cells may be provided in a cream or in dry form along with suitable stabilizers. Such cells may further express additional polypeptides, such as those mentioned, above.

Examples are given below of preferred uses of the polypeptides or compositions of the invention. The dosage of the polypeptide composition of the invention and other conditions under which the composition is used may be determined on the basis of methods known in the art.

Above composition is suitable for use in liquefaction, saccharification, and/or fermentation process, preferably in starch conversion, especially for producing syrup and fermentation products, such as ethanol.

### Use

The present invention is also directed to use of a polypeptide or composition of the present invention in a liquefaction, a saccharification and/or a fermentation process. The polypeptide or composition may be used in a single process, for example, in a liquefaction process, a saccharification process, or a fermentation process. The polypeptide or composition may also be used in a combination of processes for example in a liquefaction and saccharification process, in a liquefaction and fermentation process, or in a saccharification and fermentation process, preferably in relation to starch conversion.

### 1. Liquefaction

As used herein, the term “liquefaction” or “liquefy” means a process by which gelatinized starch is converted to less viscous liquid containing shorter chain soluble dextrins, liquefaction-inducing and/or saccharifying enzymes optionally may be added. In some embodiments, the starch substrate prepared is slurried with water. The starch slurry may contain starch as a weight percent of dry solids of about 10-55%, about 20-45%, about 30-45%, about 30-40%, or about 30-35%. Alpha-Amylase (EC 3.2.1.1) may be added to the slurry, with a metering pump, for example. The alpha-amylase typically used for this application is a thermal stable, bacterial alpha-amylase, such as a Geobacillus stearothermophilus alpha-amylase, Cytophagy alpha-amylase, etc, for example Spezyme® RSL (DuPont product), Spezyme AA (DuPont product), Spezyme® Fred (DuPont product), Clearflow AA (DuPont product), Spezyme Alpha PF (DuPont product), Spezyme Powerliq (DuPont product) can be used here.

The slurry of starch plus the alpha-amylase may be pumped continuously through a jet cooker, which is steam heated to 80-110° C., depending upon the source of the starch containing feedstock. Gelatinization occurs rapidly under these conditions, and the enzymatic activity, combined with the significant shear forces, begins the hydrolysis of the starch substrate. The residence time in the jet cooker is brief The partially gelatinized starch may then be passed into a series of holding tubes maintained at 105-110° C. and held for 5-8 min. to complete the gelatinization process (“primary liquefaction”). Hydrolysis to the required DE is completed in holding tanks at 85-95° C. or higher temperatures for about 1 to 2 hours (“secondary liquefaction”). The slurry is then allowed to cool to room temperature. This cooling step can be 30 minutes to 180 minutes, e.g. 90 minutes to 120 minutes. The liquefied starch typically is in the form of a slurry having a dry solids content (w/w) of about 10-50%; about 10-45%; about 15-40%; about 20-40%; about 25-40%; or about 25-35%.

In conventional enzymatic liquefaction process, thermostable alpha-amylase is added and the long chain starch is degraded into branched and linear shorter units (maltodextrins), but glucoamylase is not added. The glucoamylase of the present invention is highly thermostable, so it is advantageous to add the glucoamylase in the liquefaction process.

### 2. Saccharification

The liquefied starch may be saccharified into a syrup rich in lower DP (e.g., DP1+DP2) saccharides, using alpha-amylases and glucoamylases, optionally in the presence of another enzyme(s). The exact composition of the products of saccharification depends on the combination of enzymes used, as well as the type of starch processed. Advantageously, the syrup obtainable using the provided glucoamylases may contain a weight percent of DP2 of the total oligosaccharides in the saccharified starch exceeding 30%, e.g., 45%-65% or 55%-65%. The weight percent of (DP1+DP2) in the saccharified starch may exceed about 70%, e.g., 75%-85% or 80%-85%.

Whereas liquefaction is generally run as a continuous process, saccharification is often conducted as a batch process. Saccharification conditions are dependent upon the nature of the liquefact and type of enzymes available. In some cases, a saccharification process may involve temperatures of about 60-65° C. and a pH of about 4.0-4.5, e.g., pH 4.3. Saccharification may be performed, for example, at a temperature between about 40° C., about 50° C., or about 55° C. to about 60° C. or about 65° C., necessitating cooling of the Liquefact. The pH may also be adjusted as needed. Saccharification is normally conducted in stirred tanks, which may take several hours to fill or empty. Enzymes typically are added either at a fixed ratio to dried solids, as the tanks are filled, or added as a single dose at the commencement of the filling stage. A saccharification reaction to make a syrup typically is run over about 24-72 hours, for example, 24-48 hours. However, it is common only to do a pre-saccharification of typically 40-90 minutes at a temperature between 30-65° C., typically about 60° C., followed by complete saccharification in a simultaneous saccharification and fermentation (SSF). The glucoamylase of the present invention is highly thermostable, so the pre-saccharification and/or saccharification of the present invention can be carried at a higher temperature than the conventional pre-saccharification and/or saccharification. In one embodiment, a process of the invention includes pre-saccharifying starch-containing material before simultaneous saccharification and fermentation (S SF) process. The pre-saccharification can be carried out at a high temperature (for example, 50-85° C., preferably 60-75° C.) before moving into SSF. Preferredly, saccharification optimally is conducted at a higher temperature range of about 30° C. to about 75° C., e.g., 45° C.-75° C. or 50° C.-75° C. By conducting the sacchanfication processs at higher temperatures, the process can be carried out in a shorter period of time or alternatively the process can be carried out using lower enzyme dosage. Furthermore, the risk of microbial contamination is reduced when carrying the liquefaction and/or sacchanfication process at higher temperature.

In a preferred aspect of the present invention, the liquefaction and/or saccharification includes sequentially or simultaneously performed liquefaction and saccharification processes.

### 3. Fermentation

The soluble starch hydrolysate, particularly a glucose rich syrup, can be fermented by contacting the starch hydrolysate with a fermenting organism typically at a temperature around 32° C., such as from 30° C. to 35° C. “Fermenting organism” refers to any organism, including bacterial and fungal organisms, suitable for use in a fermentation process and capable of producing desired a fermentation product. Especially suitable fermenting organisms are able to ferment, i.e., convert, sugars, such as glucose or maltose, directly or indirectly into the desired fermentation product. Examples of fermenting organisms include yeast, such as Saccharomyces cerevisiae and bacteria, e.g., Zymomonas mobilis, expressing alcohol dehydrogenase and pyruvate decarboxylase. The ethanologenic microorganism can express xylose reductase and xylitol dehydrogenase, which convert xylose to xylulose. Improved strains of ethanologenic microorganisms, which can withstand higher temperatures, for example, are known in the art and can be used. See Liu et al. (2011) Sheng Wu Gong Cheng Xue Bao 27:1049-56. Commercially available yeast includes, e.g., Red Star™/Lesaffre Ethanol Red (available from Red Star/Lesaffre, USA) FALI (available from Fleischmann's Yeast, a division of Burns Philp Food Inc., USA), SUPERSTART (available from Alltech), GERT STRAND (available from Gert Strand AB, Sweden) and FERMIOL (available from DSM Specialties). The temperature and pH of the fermentation will depend upon the fermenting organism. Microorganisms that produce other metabolites, such as citric acid and lactic acid, by fermentation are also known in the art. See, e.g., Papagianni (2007) Biotechnol. Adv. 25:244-63; John et al. (2009) Biotechnol. Adv. 27:145-52.

The saccharification and fermentation processes may be carried out as an SSF process. An SSF process can be conducted with fungal cells that express and secrete glucoamylase continuously throughout SSF. The fungal cells expressing glucoamylase also can be the fermenting microorganism, e.g., an ethanologenic microorganism. Ethanol production thus can be carried out using a fungal cell that expresses sufficient glucoamylase so that less or no enzyme has to be added exogenously. The fungal host cell can be from an appropriately engineered fungal strain. Fungal host cells that express and secrete other enzymes, in addition to glucoamylase, also can be used. Such cells may express amylase and/or a pullulanase, phytase, alpha-glucosidase, isoamylase, beta-amylase cellulase, xylanase, other hemicellulases, protease, beta-glucosidase, pectinase, esterase, redox enzymes, transferase, or other enzymes. Fermentation may be followed by subsequent recovery of ethanol.

### 4. Raw Starch Hydrolysis

The present invention provides a use of the glucoamylase of the invention for producing glucoses and the like from raw starch or granular starch. Generally, glucoamylase of the present invention either alone or in the presence of an alpha-amylase can be used in raw starch hydrolysis (RSH) or granular starch hydrolysis (GSH) process for producing desired sugars and fermentation products. The granular starch is solubilized by enzymatic hydrolysis below the gelatinization temperature. Such “low-temperature” systems (known also as “no-cook” or “cold-cook”) have been reported to be able to process higher concentrations of dry solids than conventional systems (e.g., up to 45%).

A “raw starch hydrolysis” process (RSH) differs from conventional starch treatment processes, including sequentially or simultaneously saccharifying and fermenting granular starch at or below the gelatinization temperature of the starch substrate typically in the presence of at least an glucoamylase and/or amylase. Starch heated in water begins to gelatinize between 50° C. and 75° C., the exact temperature of gelatinization depends on the specific starch. For example, the gelatinization temperature may vary according to the plant species, to the particular variety of the plant species as well as with the growth conditions. In the context of this invention the gelatinization temperature of a given starch is the temperature at which birefringence is lost in 5% of the starch granules using the method described by Gorinstein. S. and Lii. C., Starch/Starke, Vol. 44 (12) pp. 461-466 (1992).

The glucoamylase of the invention may also be used in combination with an enzyme that hydrolyzes only alpha-(1, 6)-glucosidic bonds in molecules comprising at least four glucosyl residues. Preferably, the glucoamylase of the invention is used in combination with pullulanase or isoamylase. The use of isoamylase and pullulanase for debranching of starch, the molecular properties of the enzymes, and the potential use of the enzymes together with glucoamylase is described in G. M. A. van Beynum et al., Starch Conversion Technology, Marcel Dekker, New York, 1985, 101-142.

In a further aspect, the invention relates to the use of the glucoamylase of the invention include conversion of starch to e.g., syrup beverage, and/or a fermentation product, including ethanol.

### 5. Fermentation Products

The term “fermentation product” means a product produced by a process including a fermentation process using a fermenting organism. Fermentation products contemplated according to the invention include alcohols (e.g., arabinitol, butanol, ethanol, glycerol, methanol, ethylene glycol, 1,3-propanediol [propylene glycol], butanediol, glycerin, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); ketones (e.g., acetone); amino acids (e.g., aspartic acid, glutamic acid, glycine, lysine, serine, and threonine); an alkane (e.g., pentane, hexane, heptane, octane, nonane, decane, undecane, and dodecane); a cycloalkane (e.g., cyclopentane, cyclohexane, cycloheptane, and cyclooctane); an alkene (e.g. pentene, hexene, heptene, and octene); gases (e.g., methane, hydrogen (H₂), carbon dioxide (CO₂), and carbon monoxide (CO)); antibiotics (e.g., penicillin and tetracycline); enzymes; vitamins (e.g., riboflavin, B₁₂, beta-carotene); and hormones. In a preferred aspect the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, high-alcohol beer, low-alcohol beer, low-calorie beer or light beer. Preferred fermentation processes used include alcohol fermentation processes, which are well known in the art. Preferred fermentation processes are anaerobic fermentation processes, which are well known in the art.

### 6. Brewing

The glucoamylases of the present invention are highly thermostable and therefore they can be used for starch hydrolysis at high temperature for making a fermented malt beverage. For example, glucoamylases of the invention can be added to a hot mash, taking advantage of the elevated temperature to increase the reaction rate and increasing the yield of fermentable sugars prior to the addition of yeast. A glucoamylase, in combination with an amylase and optionally a pullulanase and/or isoamylase, assist in converting the starch into dextrins and fermentable sugars, lowering the residual non-fermentable carbohydrates in the final beer. The glucoamylases of the invention is added in effective amounts which can be easily determined by the skilled person in the art.

Processes for making beer are well known in the art. See, e.g., Wolfgang Kunze (2004) “Technology Brewing and Malting,” Research and Teaching Institute of Brewing, Berlin (VLB), 3rd edition. Briefly, the process involves: (a) preparing a mash, (b) filtering the mash to prepare a wort, and (c) fermenting the wort to obtain a fermented beverage, such as beer.

The brewing composition comprising a glucoamylase, in combination with an amylase and optionally a pullulanase and/or isoamylase, may be added to the mash of step (a) above, i.e., during the preparation of the mash. Alternatively, or in addition, the brewing composition may be added to the mash of step (b) above, i.e., during the filtration of the mash. Alternatively, or in addition, the brewing composition may be added to the wort of step (c) above, i.e., during the fermenting of the wort.

All references cited herein are herein incorporated by reference in their entirety for all purposes. In order to further illustrate the compositions and methods, and advantages thereof, the following specific examples are given with the understanding that they are illustrative rather than limiting.

### EXAMPLES

### Example 1

### Sequence of Penicillium russellii glucoamylase (PruGA1)

A Penicillium russellii strain was selected as a potential source for various enzymes, useful for industrial applications. The entire genome of the Penicillium russellii strain was sequenced and the nucleotide sequence of a putative glucoamylases, designated “PruGA1” was identified by sequence identity. The gene encoding PruGA1 is set forth as SEQ ID NO:1:

ATGCGCTATACTCTTTTAACGAGTATTGCCAGCGTCCTTAGCGTGGGGCCGCTGG
CATCTGCGAGCCCTACATCGAAGGATGGAAATTTGGCATCCTATATAGCGAAAG
AGGGACAACGATCTATTGTGGGGATCACTGAAAACCTCGGTGGTAAAGGCAGCA
AGACACCGGGCACGGCCGCTGGCTTATTCATTGCTAGTCCTAATATGGCGAACCC
CAATTACTATTATACGTGGACTCGTGATTCGGCCCTTACATTCAAATGCTTGATCG
ACCTGTTTGAAACTTCTGATCAGGACTATATCAGTCGTAAAGAGTTGGAAACCGA
CATTCGGAATTATGTCTCATCACAAGCAGTGCTGCAGAATGTATCCAATCCATCT
GGGACTCTGAAAGATGGCTCTGGACTCGGTGAGCCCAAGTTTGAGATCGACCTG
AACCCATTCAGTGGTTCCTGGGGCCGGCCTCAGCGTGACGGTCCCGCCCTGCGAG
CGACCGCCATGATCACTTACGCAGACTGGTTGGTTTCCCACGGTCAGAAATCCGA
AGCCACCAACATCATGTGGCCCATCATTGCAAATGACTTGGCATATGTAGGCCAG
TACTGGAACAAAACTGGATTCGACCTCTGGGAAGAGGTAGATGGATCTTCCTTTT
ACACCTTGGCTGTCCAACACCGAGCACTGGTCCAGGGAGCAAGCCTTGCGAAGA
AGCTTGGCAAGTCGTGCACTGCTTGTGTATCCCAAGCACCTCAGATTCTTTGCTTT
CTCCAGAGCTTCTGGAACGGAAATTACATCACCGCCAACATCAATCTTGACACAA
GTCGCTCCGGTATCGATTTGAACTCGATCTTGGGAAGCATCCACACCTTTGATCC
CGAAGCTTCATGTGATGATTCGACCTTCCAACCTTGCTCAGCCAGAGCGCTTGCA
AACCATAAGGTCTATGTTGACGCGTTCCGTTCAATCTATGGTGTCAATGCTGGCC
TTTCAAACGGGACAGCCGCCAATGTCGGTCGGTACCCAGAAGATGTTTACCAAG
GAGGTAACCCATGGTATCTCGCCACTCTAGCAGCAGCTGAACTGCTGTACGATGC
TCTATACCAATGGAACCAAATTGGCAAGCTCGATGTCACCAAGACCTCGCTAGCG
TTCTTCAAAGATTTTGACGCGGCCGTCAAAACAGGCACATACTCAGCGCATAGCT
CAGCCTACAGGACTCTCACCTCAGCCATCCGAACTTATGCAGATGATTTCATCAG
TCTCGTCCAACACTATACTCCTTCTAATGGGTCCTTGGCCGAGCAATATGATCGG
GATACTGGTATCCCACTGTCAGCCAATGATCTAACTTGGTCTTACGCTTCCTTCAT
CACAGCTATCGAGCGTCGTGCTTCCGTCGTGCCCGCCTCATGGGGCGAAAAATCT
GCAAATGTGGTTCCCACTACCTGCTCAGCCTCTCCTGTCACTGGAACTTACGTAG
CTGCTACTTCAGTATTCCCGACAACCACTGGATGTGTTCCGGCGACAAGCATCGT
TCCGATCACATTCTACTTGACTGAGAGCACATTCTATGGAGAAAATGTCTACATG
ACTGGCAACATTAGTGCACTTGGCAACTGGGACACGAGCAGTGGTTTCCCCCTCA
CAGCAAACTTATACACTGATTCAGACCATTTGTGGTTTGCCAGTGTTGAGCTTGTT
CCGGCGGGGACACCGTTTGAATACAAATACTACAAGGTAGAGCCGAATGGTACT
GTCATTTGGGAGAACGGTGAGAACAGGGTATACGTTGCTCCTACTGGGTGTCCGA
TCCAGCCTAGCCAAACCGATATTTGGCGT

The amino acid sequence of the PruGA1 precursor protein is set forth as SEQ ID NO: 2:

MRYTLLTSIASVLSVGPLASASPTSKDGNLASYIAKEGQRSIVGITENLG
GKGSKTPGTAAGLFIASPNMANPNYYYTWTRDSALTFKCLIDLFETSDQD
YISRKELETDIRNYVSSQAVLQNVSNPSGTLKDGSGLGEPKFEIDLNPFS
GSWGRPQRDGPALRATAMITYADWLVSHGQKSEATNIMWPIIANDLAYVG
QYWNKTGFDLWEEVDGSSFYTLAVQHRALVQGASLAKKLGKSCTACVSQA
PQILCFLQSFWNGNYITANINLDTSRSGIDLNSILGSIHTFDPEASCDDS
TFQPCSARALANHKVYVDAFRSIYGVNAGLSNGTAANVGRYPEDVYQGGN
PWYLATLAAAELLYDALYQWNQIGKLDVTKTSLAFFKDFDAAVKTGTYSA
HSSAYRTLTSAIRTYADDFISLVQHYTPSNGSLAEQYDRDTGIPLSANDL
TWSYASFITAIERRASVVPASWGEKSANVVPTTCSASPVTGTYVAATSVF
PTTTGCVPATSIVPITFYLTESTFYGENVYMTGNISALGNWDTSSGFPLT
ANLYTDSDHLWFASVELVPAGTPFEYKYYKVEPNGTVIWENGENRVYVAP
TGCPIQPSQTDIWR

The amino acid sequence of the mature form of PruGA1 confirmed by N-teminal Edman degradation is set forth as SEQ ID NO: 3:

SKDGNLASYIAKEGQRSIVGITENLGGKGSKTPGTAAGLFIASPNMANPN
YYYTWTRDSALTFKCLIDLFETSDQDYISRKELETDIRNYVSSQAVLQNV
SNPSGTLKDGSGLGEPKFEIDLNPFSGSWGRPQRDGPALRATAMITYADW
LVSHGQKSEATNIMWPIIANDLAYVGQYWNKTGFDLWEEVDGSSFYTLAV
QHRALVQGASLAKKLGKSCTACVSQAPQILCFLQSFWNGNYITANINLDT
SRSGIDLNSILGSIHTFDPEASCDDSTFQPCSARALANHKVYVDAFRSIY
GVNAGLSNGTAANVGRYPEDVYQGGNPWYLATLAAAELLYDALYQWNQIG
KLDVTKTSLAFFKDFDAAVKTGTYSAHSSAYRTLTSAIRTYADDFISLVQ
HYTPSNGSLAEQYDRDTGIPLSANDLTWSYASFITAIERRASVVPASWGE
KSANVVPTTCSASPVTGTYVAATSVFPTTTGCVPATSIVPITFYLTESTF
YGENVYMTGNISALGNWDTSSGFPLTANLYTDSDHLWFASVELVPAGTPF
EYKYYKVEPNGTVIWENGENRVYVAPTGCPIQPSQTDIWR

### Example 2

### Expression and Purification of Penicillium russellii glucoamylase (PruGA1)

The nucleotide sequence of the PruGA1 gene from Penicillium russellii synthesized and is set forth as SEQ ID NO: 4:

ATGCGCTATACTCTTTTAACGAGTATTGCCAGCGTCCTTAGCGTGGGGCCGCTGG
CATCTGCGAGCCCTACATCGAAGGATGGAAATTTGGCATCCTATATAGCGAAAG
AGGGACAACGATCTATTGTGGGGATCACTGAAAACCTCGGTGGTAAAGGCAGCA
AGACACCGGGCACGGCCGCTGGCTTATTCATTGCTAGTCCTAATATGGCGAACCC
CAATTACTATTATACGTGGACTCGTGATTCGGCCCTTACATTCAAATGCTTGATCG
ACCTGTTTGAAACTTCTGATCAGGACTATATCAGTCGTAAAGAGTTGGAAACCGA
CATTCGGAATTATGTCTCATCACAAGCAGTGCTGCAGAATGTATCCAATCCATCT
GGGACTCTGAAAGATGGCTCTGGACTCGGTGAGCCCAAGTTTGAGATCGACCTG
AACCCATTCAGTGGTTCCTGGGGCCGGCCTCAGCGTGACGGTCCCGCCCTGCGAG
CGACCGCCATGATCACTTACGCAGACTGGTTGGTTTCCCACGGTCAGAAATCCGA
AGCCACCAACATCATGTGGCCCATCATTGCAAATGACTTGGCATATGTAGGCCAG
TACTGGAACAAAACTGGATTCGACCTCTGGGAAGAGGTAGATGGATCTTCCTTTT
ACACCTTGGCTGTCCAACACCGAGCACTGGTCCAGGGAGCAAGCCTTGCGAAGA
AGCTTGGCAAGTCGTGCACTGCTTGTGTATCCCAAGCACCTCAGATTCTTTGCTTT
CTCCAGAGCTTCTGGAACGGAAATTACATCACCGCCAACATCAATCTTGACACAA
GTCGCTCCGGTATCGATTTGAACTCGATCTTGGGAAGCATCCACACCTTTGATCC
CGAAGCTTCATGTGATGATTCGACCTTCCAACCTTGCTCAGCCAGAGCGCTTGCA
AACCATAAGGTCTATGTTGACGCGTTCCGTTCAATCTATGGTGTCAATGCTGGCC
TTTCAAACGGGACAGCCGCCAATGTCGGTCGGTACCCAGAAGATGTTTACCAAG
GAGGTAACCCATGGTATCTCGCCACTCTAGCAGCAGCTGAACTGCTGTACGATGC
TCTATACCAATGGAACCAAATTGGCAAGCTCGATGTCACCAAGACCTCGCTAGCG
TTCTTCAAAGATTTTGACGCGGCCGTCAAAACAGGCACATACTCAGCGCATAGCT
CAGCCTACAGGACTCTCACCTCAGCCATCCGAACTTATGCAGATGATTTCATCAG
TCTCGTCCAACACTATACTCCTTCTAATGGGTCCTTGGCCGAGCAATATGATCGG
GATACTGGTATCCCACTGTCAGCCAATGATCTAACTTGGTCTTACGCTTCCTTCAT
CACAGCTATCGAGCGTCGTGCTTCCGTCGTGCCCGCCTCATGGGGCGAAAAATCT
GCAAATGTGGTTCCCACTACCTGCTCAGCCTCTCCTGTCACTGGAACTTACGTAG
CTGCTACTTCAGTATTCCCGACAACCACTGGATGTGTTCCGGCGACAAGCATCGT
TCCGATCACATTCTACTTGACTGAGAGCACATTCTATGGAGAAAATGTCTACATG
ACTGGCAACATTAGTGCACTTGGCAACTGGGACACGAGCAGTGGTTTCCCCCTCA
CAGCAAACTTATACACTGATTCAGACCATTTGTGGTTTGCCAGTGTTGAGCTTGTT
CCGGCGGGGACACCGTTTGAATACAAATACTACAAGGTAGAGCCGAATGGTACT
GTCATTTGGGAGAACGGTGAGAACAGGGTATACGTTGCTCCTACTGGGTGTCCGA
TCCAGCCTAGCCAAACCGATATTTGGCGTTAA

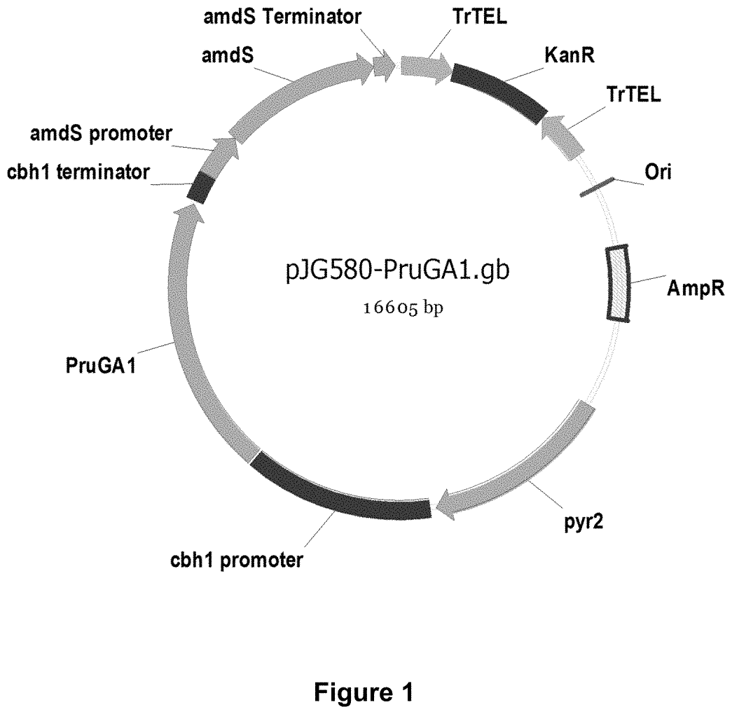

The DNA sequence of PruGA1 was optimized for expression of PruGA1 in Trichoderma reesei and inserted into the pTrex3gM expression vector (described in U.S. Published Application 2011/0136197 A1), resulting in pJG580 (FIG. 1).

The plasmid pJG580 was transformed into a Trichoderma reesei strain (described in WO 05/001036) using protoplast transformation (Te'o et al., J. Microbiol. Methods 51:393-99, 2002). The transformants were selected and fermented by the methods described in WO 2016/138315. Supernatants from these cultures were used to confirm the protein expression by SDS-PAGE analysis and assay for enzyme activity.

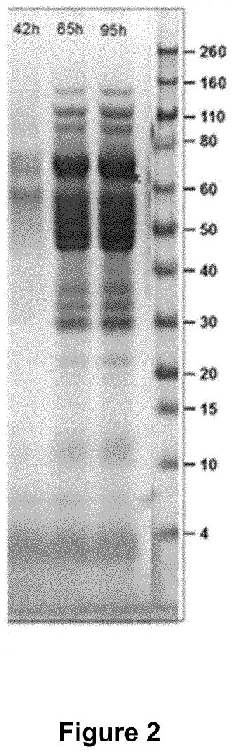

A seed culture of the transformed cells mentioned above, was subsequently grown in a 2.8 L fermenter in a defined medium. Fermentation broth was sampled at fermentation times of 42, 65, and 95 hours to run samples on SDS-PAGE, measurements of dry cell weight, residual glucose, and extracellular protein concentration. FIG. 2 shows the production profile of PruGA1 at 95-hour fermentation. After 95 hours post fermentation, following centrifugation, filtration and concentration, 500 mL of the concentrated sample was obtained. The protein concentration was determined to be 10.7 g/L by the BCA method.

Two hundred mL of clarified culture broth was loaded onto a 20-mL beta-cyclodextrin coupled Sepharose 6 column (pre-equilibrated with 20 mM sodium acetate pH 5.0, 150 mM NaCl), followed by washing with 3 column volumes of the same buffer. Elution was performed using 5 column volumes of 10 mM alpha-cyclodextrin in 20 mM sodium acetate pH 5.0 containing 150 mM NaCl. Fractions were collected and assayed for glucoamylase activity and run on SDS-PAGE. The fractions containing the target protein were pooled, and concentrated using Amicon Ultra-15 device with 10 K MWCO using 20 mM sodium acetate pH 5.0 containing 150 mM NaCl. The purified sample is above 99% pure and stored in 40% glycerol at −80° C. until usage.

### Example 3

### Specific Activity of PruGA1 on Soluble Starch

Glucoamylase specific activity was assayed based on the release of glucose by glucoamylase from soluble starch using a coupled glucose oxidase/peroxidase (GOX/HRP) method (Anal. Biochem. 105 (1980), 389-397).

Substrate solutions were prepared by mixing 9 mL of soluble starch (1% in water, w/w) and 1 mL of 0.5 M pH 5.0 sodium acetate buffer in a 15-mL conical tube. Coupled enzyme (GOX/HRP) solution with ABTS was prepared in 50 mM sodium acetate buffer (pH 5.0), with the final concentrations of 2.74 mg/mL ABTS, 0.1 U/mL HRP, and 1 U/mL GOX.

Serial dilutions of glucoamylase samples and glucose standard were prepared in purified water. Each glucoamylase sample (10 μL) was transferred into a new microtiter plate (Coming 3641) containing 90 μL of substrate solution preincubated at 50° C. for 5 min at 600 rpm. The reactions were carried out at 50° C. for 10 min with shaking (600 rpm) in a thermomixer (Eppendorf), 10 μL of reaction mixtures as well as 10 μL of serial dilutions of glucose standard were quickly transferred to new microtiter plates (Corning 3641), respectively, followed by the addition of 100 μL of ABTS/GOX/HRP solution. Absorbance at 405 nm was immediately measured at 11 seconds intervals for 5 min using a SoftMax Pro plate reader (Molecular Device). The output was the reaction rate, Vo, for each enzyme concentration. Linear regression was used to determine the slope of the plot Vo vs. enzyme dose. The specific activity of glucoamylase was calculated based on the glucose standard curve using Equation 1:

Specific Activity (Unit/mg)=Slope (enzyme)/slope (std)×1000 (1),

where 1 Unit=1 μmol glucose/min.

Using the method mentioned above, specific activity of PruGA1 was determined and compared with the benchmark, AnGA (a glucoamylase from Aspergillus niger). Results are shown in Table 2. The PruGA1 specific activity of 197 U/mg towards soluble starch was approximately 2 fold higher than the other glucoamylase AnGA.

TABLE 2
Specific activity of purified PruGA1 towards
soluble starch compared with AnGA

| | Specific activity (U/mg/min) | |
|---|---|---|
| Substrate | PruGA1 | AnGA |
| Soluble Starch | 197 | 106 |

### Example 4

### Pullulan-Hydrolyzing Activity of Glucoamylase PruGA1

Glucoamylase activity towards pullulan was assayed using the same protocol as described above for specific activity of glucoamylase PruGA1 towards soluble starch, except that the enzymes was dosed at 10 ppm. Table 3 summarizes pullulan-hydrolyzing activities of PruGA1 and the benchmark, AnGA. The activity of PruGA1 on pullulan was approximately 6 times as high as that of AnGA.

TABLE 3
Pullulan-hydrolyzing activity of PruGA1 compared with AnGA.

| | GA (dosed at 10 ppm) activity | |
|---|---|---|
| | towards pullulan | |
| Substrate | PruGA1 | AnGA |
| pullulan | 154 | 24 |

### Example 5

### pH and Temperature Effect on PruGA1 glucoamylase Activity

The effect of pH (from 2.0 to 10.0) on PruGA1 activity was monitored using soluble starch (1% in water, w/w) as substrate. Buffer working solutions consisted of the combination of glycine/sodium acetate/HEPES (250 mM), with pH varying from 2.0 to 10.0. Substrate solutions were prepared by mixing soluble starch (1% in water, w/w) with 250 mM buffer solution at a ratio of 9:1. Enzyme working solutions were prepared in water at a certain dose (showing signal within linear range as per dose response curve). All the incubations were carried out at 50° C. for 10 min following the same protocol as described above for specific activity of glucoamylase PruGA1 towards soluble starch. Enzyme activity at each pH was reported as relative activity compared to enzyme activity at optimum pH. The pH profile of PruGA1 is shown in Table 4. PruGA1 was found to have an optimum pH at about 5.0 and retains greater than 70% of maximum activity between pH 2.8 and 7.0.

TABLE 4
pH profile of PruGA1

| pH | Relative activity (%) |
|---|---|
| 2 | 31 |
| 2.5 | 48 |
| 3 | 81 |
| 3.5 | 92 |
| 4 | 98 |
| 5 | 100 |
| 6 | 98 |
| 7 | 71 |
| 8 | 32 |
| 9 | 13 |
| 10 | 1 |

The effect of temperature (from 40° C. to 84° C.) on PruGA1 activity was monitored using soluble starch (1% in water, w/w) as substrate. Substrate solutions were prepared by mixing 9 mL of soluble starch (1% in water, w/w) and 1 mL of 0.5 M buffer (pH 5.0 sodium acetate) into a 15-mL conical tube. Enzyme working solutions were prepared in water at a certain dose (showing signal within linear range as per dose response curve). Incubations were performed at temperatures from 40° C. to 84° C., respectively, for 10 min following the same protocol as described above for specific activity of glucoamylase PruGA1 towards soluble starch. Activity at each temperature was reported as relative activity compared to enzyme activity at optimum temperature. The temperature profile of PruGA1 is shown in Table 5. PruGA1 displayed optimal activity at 75° C. and activity remained above 70% of maximum activity between 63° C. and 79° C.

TABLE 5
Temperature-activity profile of PruGA1

| Temp. (° C.) | Relative activity (%) |
|---|---|
| 40 | 24 |
| 44.7 | 34 |
| 49.4 | 42 |
| 55 | 54 |
| 59.7 | 63 |
| 65 | 77 |
| 69.2 | 90 |
| 74.6 | 100 |
| 80 | 58 |
| 85 | 19 |

### Example 6

### Saccharification Performance of PruGA1 at pH 4.5 at Different Temperatures

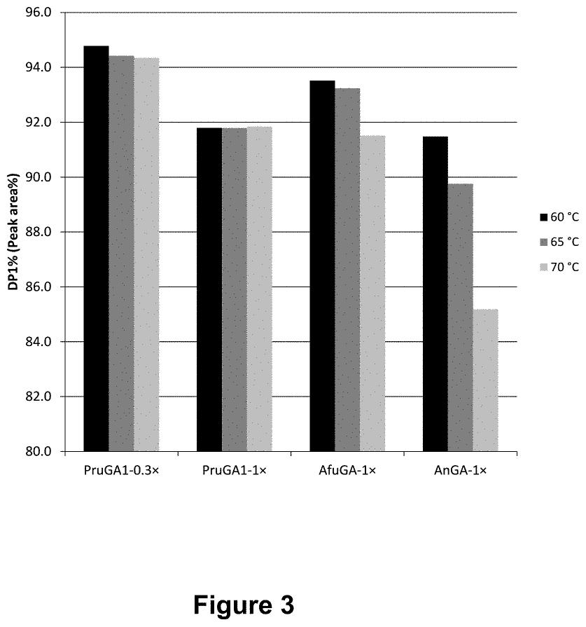

The activity of PruGA1, AnGA and AfuGA (described in W02014092960) was evaluated under saccharification conditions at pH 4.5 with different incubation temperatures. The evaluation of DP1 was measured by analyzing sugar compositions with equal enzyme dosage. Alpha-amylase-pretreated corn starch liquefact (prepared at 34.9% ds, pH 3.8) was used as a starting substrate. The incubations of glucoamylases (dosed at 0.121 mg/gds as lxdose) and corn starch liquefact (34% ds) were performed at pH 4.5 at 60, 65, and 70° C., respectively. Samples were collected at 16, 24, 40, 64, and 72 h, respectively. All the incubations were quenched by heating at 100° C. for 15 min. Sample supernatant was transferred and diluted 400-fold in 5 mM H₂SO₄ for HPLC analysis using an Agilent 1200 series system with a Fast fruit column (100 mm×7.8 mm) run at 85° C. 10 μL samples were loaded on the column and separated with an isocratic gradient of purified water as the mobile phase at a flow rate of 1.0 mL/min. The oligosaccharide products were detected using a refractive index detector. The glucogenic activities of the samples are summarized in Table 6. Selecting DP1% after 72-h incubation (FIG. 3) as an example, PruGA1 at 0.3×dosage (40 μg/gds) outperformed AfuGA-1×dosage (121 μg/gds) and AnGA-1×dosage (121 μg/gds) at all three tested temperatures. Even when the incubation temperature was increased up to 70° C., PruGA1 maintained its superior performance in terms of DP1 production. The glucogenic activities of the samples are summarized in Table 6.

TABLE 6
DP1 production of PruGA1, AfuGA, and AnGA
(1× dosed set at 121 μg/gds) incubated with corn
starch liquefact at pH 4.5, at various temperatures.

| | Time | | | |
|---|---|---|---|---|
| Sample name | (h) | 60° C. | 65° C. | 70° C. |
| PruGA1-0.3× | 16 | 74.2 | 77.7 | 80.6 |
| | 24 | 84.2 | 85.6 | 87.2 |
| | 40 | 91.2 | 93.0 | 93.5 |
| | 64 | 95.3 | 95.1 | 95.0 |
| | 72 | 94.8 | 94.4 | 94.3 |
| PruGA1-1× | 16 | 93.7 | 94.3 | 94.2 |
| | 24 | 94.7 | 94.2 | 93.5 |
| | 40 | 93.5 | 92.7 | 92.5 |
| | 64 | 92.3 | 92.3 | 92.2 |
| | 72 | 91.8 | 91.8 | 91.8 |
| AfuGA-1× | 16 | 85.9 | 86.9 | 86.3 |
| | 24 | 88.6 | 89.3 | 88.6 |
| | 40 | 91.6 | 91.8 | 90.9 |
| | 64 | 93.9 | 93.8 | 92.3 |
| | 72 | 93.5 | 93.2 | 91.5 |
| AnGA-1× | 16 | 82.3 | 83.0 | 81.8 |
| | 24 | 85.5 | 85.4 | 82.9 |
| | 40 | 88.9 | 87.9 | 84.2 |
| | 64 | 91.7 | 89.8 | 85.4 |
| | 72 | 91.5 | 89.8 | 85.2 |

### Example 7

### PruGA1 Saccharification Evaluation at pH 5.5 and 70° C.

The glucogenic activity of PruGA1 at an elevated temperature (aiming to shorter saccharification time) was evaluated. Corn starch liquefact (32% ds, pH 3.9) was obtained from alpha-amylase-pretreated corn starch liquefact. The incubations of PruGA1 with different dosages and corn starch liquefact (32% ds) were performed at pH 5.5, 70° C. Samples were collected at 18, 26, 42, 50, 66, and 72 h. All the incubations were quenched by heating at 100° C. for 15 min. Supernatant of the sample was transferred and diluted 400-fold in 5 mM H2504 for HPLC analysis using same conditions as shown in Example 6. The glucogenic activities of the samples were summarized in Table 7. The results showed that PruGA1 (dosed at 30 μg/gds) could reach>95% of DP1 production after two days incubation at pH 5.5, 70° C.

TABLE 7
Sugar production by PruGA1 at different dosages
(from 20 to 50 μg/gds) under sacharification condition
at pH 5.5 and 70° C. using corn starch liquefact as a substrate

| Enzyme | Incubation | | | | |
|---|---|---|---|---|---|
| dose | time (h) | DP3+% | DP3% | DP2% | DP1% |
| 50 μg/gds | 18 | 8.2 | 0.6 | 2.2 | 89.0 |
| | 26 | 2.4 | 0.4 | 2.3 | 94.9 |
| | 42 | 0.6 | 0.4 | 3.3 | 95.8 |
| | 50 | 0.9 | 0.4 | 3.6 | 95.1 |
| | 66 | 1.3 | 0.5 | 4.0 | 94.2 |
| | 72 | 1.4 | 0.5 | 4.3 | 93.9 |
| 40 μg/gds | 18 | 12.4 | 0.6 | 1.9 | 85.1 |
| | 26 | 5.6 | 0.4 | 1.9 | 92.0 |
| | 42 | 1.0 | 0.2 | 2.8 | 96.0 |
| | 50 | 1.1 | 0.4 | 3.1 | 95.4 |
| | 66 | 1.4 | 0.4 | 3.7 | 94.4 |
| | 72 | 1.3 | 0.4 | 4.0 | 94.3 |
| 30 μg/gds | 18 | 16.8 | 0.8 | 3.0 | 79.3 |
| | 26 | 10.7 | 0.5 | 1.8 | 87.0 |
| | 42 | 2.9 | 0.4 | 2.3 | 94.4 |
| | 50 | 2.0 | 0.3 | 2.6 | 95.1 |
| | 66 | 1.6 | 0.4 | 3.1 | 94.9 |
| | 72 | 1.3 | 0.4 | 3.3 | 95.0 |
| 20 μg/gds | 18 | 24.3 | 0.6 | 7.4 | 67.7 |
| | 26 | 17.3 | 0.7 | 3.4 | 78.6 |
| | 42 | 8.2 | 0.5 | 1.7 | 89.6 |
| | 50 | 6.5 | 0.5 | 2.0 | 91.0 |
| | 66 | 3.8 | 0.4 | 2.3 | 93.5 |
| | 72 | 3.3 | 0.4 | 2.4 | 93.9 |

### Example 8

### Raw Starch Activity of PruGA1

The activity of PruGA1 on raw starch assay was measured and compared to the activity of Trichoderma reesei glucoamylase (TrGA) using for granular starch hydrolyzing enzyme (GSHE) fermentation and direct starch to glucose/maltose process (DSTG/DSTM). Alpha-amylase and glucoamylase were blended at a ratio of 1:6.6 in this assay. The Aspergillus kawachii amylase (AkAA, described in W02013169645) was used. A Fast Fruit HPLC column (Waters) was used for sugar profile analysis and glucose (final product) was used to determine enzyme raw starch hydrolyzing capability.

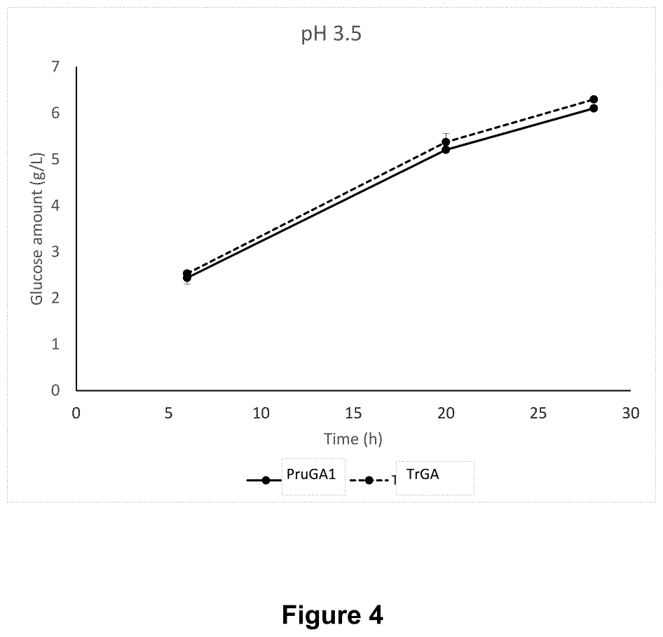

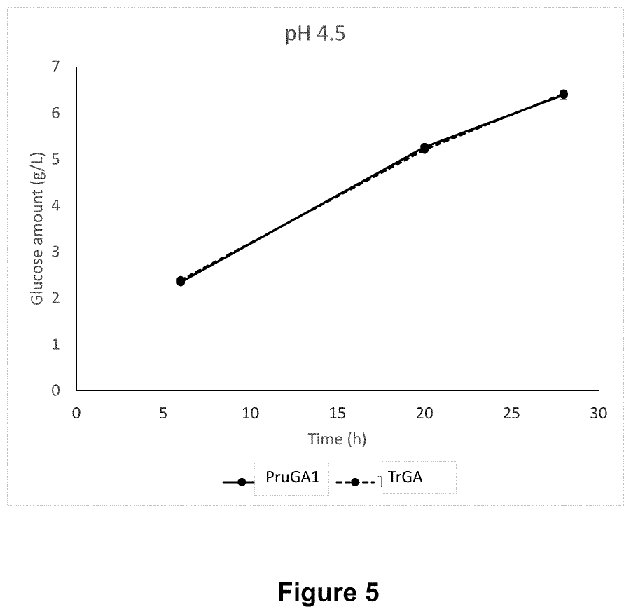

150 μL of the corn starch substrate (1%, in 50 mM pH 3.5/pH 4.5 sodium acetate buffer) was dispensed into 0.5 mL microtiter plates using wide bore tips. 10 μL of amylase and 10 μL of glucoamylase were added per well to set final dosages for AkAA and glucoamylase were 1.5 ppm and 10 ppm, respectively. The samples were placed in iEMS incubator set at 32° C., 900 rpm for 6, 20 and 28 h. 50 μL of 0.5 M NaOH was added to quench the reactions and the starch plug was suspended by putting the plate on a shaker for 2 min. After that, the plate was sealed and centrifuged at 2500 rpm for 3 min. For HPLC analysis, the supernatant was diluted 10 fold using 0.01 N H₂SO₄. 10 μL samples of samples were analyzed using a Agilent 1200 series HPLC equipped with a refractive index detector. The column used was a Phenomenex Rezex-RFQ Fast Fruit column (cat #00D-0223-K0) with a Phenomenex Rezex ROA Organic Acid guard column (cat #03B-0138-K0). The mobile phase was 0.01 N H₂SO₄, and the flow rate was 1.0 mL/min at 85° C. The results are shown on FIGS. 4 and 5. PruGA1 exhibited comparable activity to benchmark glucoamylase TrGA both at pH 3.5 and pH 4.5 towards raw starch.

### Example 9

### Evaluation of PruGA1 for Low pH Fermentation

The glucogenic activity of PruGA1 under low pH fermentation conditions was evaluated. The performances of PruGA1 and TrGA were tested at equal protein concentration of 0.25 mg/gds. Amylase-treated corn starch liquefact (34.9% ds, pH 3.8) was used as substrate. The pH of corn starch liquefact (32% ds, amylase pre-treated) was adjusted to pH 3.0 and 10 g was transferred to a 50 mL glass bottle. The incubations were performed at 32° C. and 55° C. Samples were collected at 17, 24, 41, 48, 63, 72 h. All the incubations were quenched by heating at 100° C. for 15 min. Supernatants of the samples were transferred and diluted 400-fold in 5 mM H₂SO₄ for HPLC analysis using the same conditions as shown in Example 7. The values reported in Table 7 reflect the peak area percentages of each DPn as a fraction of the total DP1, DP2, DP3, and DP3+. The data in Table 8 show that PruGA1 exhibited higher glucogenic activity than TrGA when dosed at equal protein concentration and incubated at 32° C., pH 3. When the incubation temperature was increased to 55° C., PruGAl hydrolyzed high DP sugars very efficiently, with only 5% DP3+ remaining after 17 h incubation, while that for TrGA was 18%.

To further evaluate performance of PruGA1 at low pH, another test was conducted towards starch liquefact at an even lower pH (pH 2.0). The screening procedure was the same as that for pH 3.0 except that the enzymes were dosed at 0.2 mg/gds and samples were collected at 4, 21, 29, 45, 53, 70 h. As shown in Table 9, at pH 2 and 32° C., the DP1 released by PruGA1 was 77.4% after 70 h, while TrGA released 54% . The percent of DP1 released by PruGA1 at 55° C. was 26.2% while only 3.2% for TrGA reaction.

TABLE 8
Sugar composition analysis of PruGAl and TrGA hydrolysis
of starch liquefact at pH 3, at 32° C. or 55° C.

| Sample | Incubation | Time | | | | |
|---|---|---|---|---|---|---|
| name | temp. (° C.) | (h) | DP3+ % | DP3% | DP2% | DP1% |
| TrGA | 32 | 17 | 36.9 | 1.2 | 6.1 | 55.8 |
| | | 24 | 24.7 | 0.6 | 3.9 | 70.8 |
| | | 41 | 20.9 | 0.6 | 1.5 | 77.1 |
| | | 48 | 18.2 | 0.5 | 1.5 | 79.8 |
| | | 63 | 15.2 | 0.2 | 1.6 | 82.9 |
| | | 72 | 14.3 | 0.4 | 1.7 | 83.6 |
| PruGA1 | 32 | 17 | 35.5 | 1.3 | 4.1 | 59.0 |
| | | 24 | 20.9 | 0.6 | 2.2 | 76.3 |
| | | 41 | 12.2 | 0.2 | 1.4 | 86.3 |
| | | 48 | 9.7 | 0.4 | 1.6 | 88.3 |
| | | 63 | 5.7 | 0.2 | 1.8 | 92.3 |
| | | 72 | 5.3 | 0.2 | 2.1 | 92.4 |
| TrGA | 55 | 17 | 18.1 | 0.6 | 2.6 | 78.7 |
| | | 24 | 14.2 | 0.5 | 2.3 | 83.1 |
| | | 41 | 9.7 | 0.4 | 2.7 | 87.3 |
| | | 48 | 8.9 | 0.4 | 2.7 | 88.0 |
| | | 63 | 7.2 | 0.0 | 3.0 | 89.8 |
| | | 72 | 6.7 | 0.2 | 3.1 | 89.9 |
| PruGA1 | 55 | 17 | 4.8 | 0.4 | 2.6 | 92.2 |
| | | 24 | 2.0 | 0.4 | 3.4 | 94.2 |
| | | 41 | 2.5 | 0.5 | 4.8 | 92.1 |
| | | 48 | 2.3 | 0.6 | 5.4 | 91.7 |
| | | 63 | 2.5 | 0.0 | 6.9 | 90.7 |
| | | 72 | 1.8 | 0.9 | 7.0 | 90.3 |

TABLE 9
Sugar composition analysis of PruGAl and TrGA hydrolysis
of starch liquefact at pH 2, at 32° C. or 55° C.

| Sample | Incubation | Time | | | | |
|---|---|---|---|---|---|---|
| name | temp. (° C.) | (h) | DP3+ % | DP3% | DP2% | DP1% |
| TrGA | 32 | 4 | 77.7 | 7.7 | 4.5 | 10.1 |
| | | 21 | 48.5 | 6.2 | 10.1 | 35.2 |
| | | 29 | 44.1 | 4.2 | 11.1 | 40.6 |
| | | 45 | 36.6 | 1.9 | 12.2 | 49.3 |
| | | 53 | 35.3 | 1.3 | 12.0 | 51.5 |
| | | 70 | 33.5 | 0.9 | 11.6 | 54.0 |
| PruGA1 | 32 | 4 | 71.6 | 7.8 | 5.7 | 14.9 |
| | | 21 | 35.3 | 0.9 | 11.6 | 52.2 |
| | | 29 | 29.4 | 0.2 | 9.4 | 61.0 |
| | | 45 | 26.0 | 0.2 | 5.0 | 68.8 |
| | | 53 | 25.2 | 0.3 | 3.4 | 71.2 |
| | | 70 | 20.3 | 0.6 | 1.7 | 77.4 |
| TrGA | 55 | 4 | 87.3 | 6.1 | 3.6 | 3.0 |
| | | 21 | 87.2 | 5.9 | 3.8 | 3.2 |
| | | 29 | 87.2 | 6.0 | 3.7 | 3.1 |
| | | 45 | 86.6 | 6.0 | 3.9 | 3.4 |
| | | 53 | 86.9 | 6.1 | 3.8 | 3.2 |
| | | 70 | 87.1 | 6.0 | 3.7 | 3.2 |
| PruGA1 | 55 | 4 | 62.1 | 8.0 | 7.2 | 22.7 |
| | | 21 | 59.2 | 7.8 | 7.8 | 25.2 |
| | | 29 | 57.8 | 7.7 | 7.9 | 26.5 |
| | | 45 | 57.2 | 7.8 | 8.1 | 26.9 |
| | | 53 | 59.2 | 7.6 | 7.7 | 25.5 |
| | | 70 | 58.1 | 7.7 | 7.9 | 26.2 |

### Example 10

### High Temperature Infusion Mashing with Glucoamylase on Wort Substrate

The glucogenic activity of PruGA1 in high temperature infusion mashing process was evaluated in comparison to other glucoamylase benchmarks for brewing application wort substrate.

The mashing operation performed with 55% Pilsner malt (Pilsner malt; Fuglsang Denmark, Batch 13.01.2016) and 45% Corn grist (Benntag Nordic; Nordgetreide GmBH Lubec, Germany, Batch: 02.05.2016.), using a water to grist ratio of 4.0:1. Pilsner malt was milled at a Buhler Miag malt mill (0.5 mm setting). Maize grist (1.35 g), Malt (milled pilsner malt, 1.65 g) was mixed in wheaton cups (wheaton glass containers with cap) preincubated with 12.0 g tap water pH adjusted to pH 5.4 with 2.5 M sulphuric acid. The resulting substrate (15% ds, pH 5.4) was then used for glucoamyalse performance evaluation. PruGA1 (10 μL of 1 mg/mL stock) was added into 90 μL of the substrate dispensed in a PCR microtiter plate (Axygen). The other glucoamylases evaluated were: TrGA variant A, a Trichorderma reesie glucoamylase variant (with the substitutions D44R and D539R, 10 μL of 2 mg/mL stock) and Aspergillus niger glucoamylase (AnGA, 10 μL of 1 mg/mL stock). All the incubations were carried out at 64° C. for 4 h, or for even higher mashing temperature, the incubations were done at 70° C. for 2 h; followed by 79° C. for 15 min. After quenching the reaction at 95° C. for 10 min, the reaction mixture was centrifuged at 3700 rpm for 10 min. Supernatant samples were transferred and diluted 20-fold in 5 mM H2SO4 for HPLC analysis. HPLC separation was performed using an Agilent 1200 series HPLC system with a Fast fruit column (100mm×7.8 mm) at 85° C. The samples (10 μL) were loaded on HPLC column and separated with an isocratic gradient of purified water as the mobile phase at a flow rate of 1.0 mL/min. The oligosaccharide products were detected using a refractive index detector. The glucogenic activities of the samples are summarized in Table 10. The 100 ppm PruGA1 sample exhibited comparable performance to the TrGA variant A (TrGA vA) glucoamylase at 200 ppm. The PruGAenzyme also showed superior performance to benchmark when the incubation temperature was increased up to 70° C. and the incubation time was shortened to 2 h.

TABLE 10
Sugar composition analysis of glucoamylases
incubated with wort substrate at pH 5.4

| Mashing | Sample | | | | |
|---|---|---|---|---|---|
| condition | name | DP3+% | DP3% | DP2% | DP1% |
| 64° C. 4 h | AnGA | 4 | 2 | 3 | 92 |
| | TrGA vA | 0 | 1 | 2 | 98 |
| | PruGA1 | 0 | 0 | 2 | 98 |
| 70° C. 2 h | TrGA vA | 15 | 2 | 38 | 44 |
| | PruGA1 | 3 | 2 | 3 | 92 |

### Example 11

### Very High Temperature Infusion Mashing Using malt and Corn with glucoamylase

The glucoamylase PruGA1 was tested in mashing operation with 55% pilsner malt (Pilsner malt; Fuglsang Denmark, Batch 13.01.2016) and 45% corn grist (Benntag Nordic; Nordgetreide GmBH Lubec, Germany, Batch: 02.05.2016.), using a water to grist ratio of 4.0:1. Pilsner malt was milled using a Buhler Miag malt mill (0.5 mm setting).

Maize grits (1.35g) and malt (milled pilsner malt, 1.65 g) was mixed in wheaton cups (wheaton glass containers with cap) preincubated with 12.0 g tap water pH adjusted to pH 5.4 with 2.5 M sulphuric acid. Glucoamylase enzyme was added based on ppm active protein (in total 1.0 ml) and water as no enzyme control. The wheaton cups were placed in Drybath (Thermo Scientific Stem station) with magnetic stirring and the following mashing program was applied; sample were heated to 64° C. for 30 minutes; maintained at 64° C. for 15 minutes; heated to 79° C. for 15 minutes by increasing temperature with 1° C./minute; maintained at 79° C. for 15 minutes; heated to 90° C. for 11 minutes by increasing temperature with 1° C./minute maintained at 90° C. for 15 minutes; cooled to 79° C. for 15 minutes and finally heated to 79° C. for 15 minutes and mashed off 10 ml sample was transferred to Falcon tubes and boiled at 100° C. for 20 minutes to ensure complete enzyme inactivation. Spend grain was separated from the wort by centrifugation in a Heraeus Multifuge X3R at 4500 rpm for 20 minutes at 10° C. Supernatant was collected for HPLC sugar analysis using standard methods The results are shown in Table 11.

TABLE 11
Relative distribution of sugars (DPI to DP5+)
in wort determined by HPLC

| | % Relative sugar | | | | | |
|---|---|---|---|---|---|---|
| | | | | | | Total |
| | DP1 | DP2 | DP3 | DP4 | DP5+ | Sugar |
| 200 ppm | 48.04 | 33.95 | 3.39 | 1.38 | 13.23 | 100.00 |
| PruGA1 | | | | | | |
| 300 ppm | 58.42 | 26.63 | 1.71 | 1.21 | 12.02 | 100.00 |
| PruGA1 | | | | | | |
| 500 ppm | 74.60 | 15.53 | 1.06 | 0.48 | 8.34 | 100.00 |
| PruGA1 | | | | | | |
| 750 ppm | 83.44 | 7.41 | 0.75 | 0.12 | 8.28 | 100.00 |
| PruGA1 | | | | | | |
| No enzyme— | 12.46 | 35.86 | 11.45 | 4.34 | 35.90 | 100.00 |
| control | | | | | | |

It is clear that PruGA1 facilitated high production of DP1 in a dose dependent manner. Up to 83.44% DP1 was produced at a dose of 750 ppm enzyme.

### Example 12

### 100% Corn Infussion Mashing at High Temperature with glucoamylase

The goal of this experiment was to evaluate the glucogenic activity of PruGA1 in high temperature mashing process using corn and malt compared to industry benchmarks. The mashing operation was performed with 100% corn grist (Benntag Nordic; Nordgetreide GmBH Lübec, Germany, Batch: 02.05.2016.), using a water to grist ratio of 4.0:1.

Corn grits (3.0 g) was added in wheaton cups (wheaton glass containers with cap) preincubated with 12.0 g tap water pH adjusted to pH 5.4 with 2.5 M sulphuric acid. Glucoamylase enzyme was added based on ppm active protein (in total 1.0 ml) or water as a no-enzyme control. A fixed concentration of 5.0 ppm alpha-amylase (AMYLEX® 5T, from Dupont) and 0.21 ppm beta-glucanase (LAMINEX® 750, from Dupont) was applied all samples to facilitate liquefaction and filterbilityThe wheaton cups were placed in Drybath (Thermo Scientific Stem station) with magnetic stirring and the three different mashing program was applied. According to Profile 1, samples were heated to 64° C.; maintained at 64° C. for 80 minutes; heated to 80° C. for 10 minutes by increasing temperature with 1.6° C/minute; maintained at 80° C. for 30 minutes and then mashed off. According to Profile 2, samples were heated to 70° C.; maintained at 70° C. for 80 minutes; heated to 80° C. for 10 minutes by increasing temperature with 1.0° C./minute; maintained at 80° C. for 30 minutes and then mashed off; According to Profile 3, samples were heated to 75° C.; maintained at 75° C. for 80 minutes; heated to 80° C. for 10 minutes by increasing temperature with 0.5° C./minute; maintained at 80° C. for 30 minutes and then mashed off 10 ml samples were transferred to Falcon tubes and boiled at 100° C. for 20 minutes to ensure complete enzyme inactivation. Spent grains was separated from the wort by centrifugation in a Heraeus Multifuge X3R at 4500 rpm for 20 minutes at 10° C. Supernatant was collected for HPLC sugar analysis. The glucogenic activities of the samples are summarized in Table 12.

TABLE 12
Sugar composition analysis of glucoamylases after infussion mashing
using 100% corn at pH 5.4 using various temperatures.

| | Sample | | | | |
|---|---|---|---|---|---|
| Mashing condition | name | DP3+% | DP3% | DP2% | DP1% |
| profile 1: 64° C. 80 min, | TrGA | 28 | 5 | 12 | 55 |
| 80° C. 30 min | PruGA1 | 32 | 9 | 22 | 37 |
| profile 2: 70° C. 80 min, | TrGA | 31 | 7 | 21 | 41 |
| 80° C. 30 min | PruGA1 | 29 | 8 | 24 | 39 |
| profile 3: 75° C. 80 min, | TrGA | 41 | 11 | 18 | 30 |
| 80° C. 30 min | PruGA1 | 26 | 9 | 25 | 40 |

PruGA1 exhibited enhanched performance at 70° C. and 75° C. mashing profiles (final concentration: 18 ppm) compared to TrGA (final concentration: 18 ppm), the wild-type from Trichorderma reesie glucoamylase.

1. A polypeptide having glucoamylase activity, selected from the group consisting of:
(a) a polypeptide comprising an amino acid sequence having at least 95%, such as even at least 96%, 97%, 98%, 99% or 100% identity to the polypeptide of SEQ ID NO: 3;
(b) a polypeptide encoded by a polynucleotide that hybridizes under at least low stringency conditions, more preferably at least medium stringency conditions, even more preferably at least medium-high stringency conditions, most preferably at least high stringency, and even most preferably at least very high stringency conditions with
(i) the mature polypeptide coding sequence of SEQ ID NO: 1,
(ii) the genomic DNA sequence comprising the mature polypeptide coding sequence of SEQ ID NO: 1, or
(iii) a full-length complementary strand of (i) or (ii);
(c) a polypeptide encoded by a polynucleotide comprising a nucleotide sequence having preferably at least 75%, more preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, 97%, 98%, 99% or 100% identity to the mature polypeptide coding sequence of SEQ ID NO: 1;
(d) a variant comprising a substitution, deletion, and/or insertion of one or more (e.g., several) amino acids of the polypeptide of SEQ ID NO: 3; and
(e) a fragment of a polypeptide of (a), (b), (c) or (d) that has glucoamylase activity.

2. A polynucleotide comprising a nucleotide sequence that encodes the polypeptide of claim 1.

3. A vector comprising the polynucleotide of claim 2 operably linked to one or more control sequences that control the production of the polypeptide in an expression host.

4. A recombinant host cell comprising the polynucleotide of claim 2.

5. The host cell of claim 4, which is an ethanologenic microorganism.

6. The host cell of claim 4, which further expresses and secretes one or more additional enzymes selected from the group comprising protease, hemicellulase, cellulase, peroxidase, lipolytic enzyme, metallolipolytic enzyme, xylanase, lipase, phospholipase, esterase, perhydrolase, cutinase, pectinase, pectate lyase, mannanase, keratinase, reductase, oxidase, phenoloxidase, lipoxygenase, ligninase, alpha-amylase, pullulanase, phytase, tannase, pentosanase, malanase, beta-glucanase, arabinosidase, hyaluronidase, chondroitinase, laccase, transferrase, or a combination thereof.

7. A method for saccharifying a starch-containing material comprising the steps of: i) contacting the starch-containing material with an alpha-amylase; and ii) contacting the starch-containing material with a glucoamylase at a temperature of at least 70° C.; wherein the method produces at least 70% free glucose from the starch-containing material (substrate).

8. The method of claim 7, wherein the step (ii) is carried out at a temperature of at least 75° C., preferably at least 80° C. for between 12 and 96 hours, preferably 18 to 60 hours.

9. The method of claim 7, wherein the glucoamylase maintains at least 70%, at least 80%, at least 85%, at least 90%, at least 95%, at least 100% of relative activity at a temperature of at least 70° C., and/or at a pH between 2.0 and 7.0, preferably between pH 4.0 and pH 6.0, more preferably between pH 4.5 and pH 5.5.

10. The method of claim 7, wherein the method includes sequentially or simultaneously performing step (i) and step (ii).

11. The method of claim 7, wherein the method further comprises a pre-saccharification before saccharification step ii).

12. The method of claim 7, wherein the glucoamylase is the polypeptide of claim 1.

13. The method of claim 7, wherein the step (i) is carried out at or below the gelatinization temperature of the starch-containing material.

14. The method of claim 7, wherein an additional debranching enzyme is absent during step (i) and/or step (ii).

15. The method of claim 14, wherein the debranching enzyme is pullulanase.

16. A saccharide produced by method of claim 7.

17. A method for producing fermentation products from the saccharide of claim 16, wherein the saccharide is fermented by a fermenting organism.

18. The method of claim 17, wherein the fermentation process is performed sequentially or simultaneously with the step (ii).

19. The method of claim 17 , wherein the fermentation product comprises ethanol.

20. The method of claim 17, wherein the fermentation product comprises a non-ethanol metabolite.

21. The method of claim 20, wherein the metabolite is citric acid, lactic acid, succinic acid, monosodium glutamate, gluconic acid, sodium gluconate, calcium gluconate, potassium gluconate, an organic acid, glucono delta-lactone, sodium erythorbate, omega 3 fatty acid, butanol, iso-butanol, an amino acid, lysine, tyrosine, threonine, glycine, itaconic acid, 1,3-propanediol, vitamins, enzymes, hormones, isoprene or other biochemicals or biomaterials.

22. A method of applying the polypeptide of claim 1 in brewing.

23. A method of applying the polypeptide of claim 1 for the production of beer or a malt-based beverage.
